(19) United States
(12) Patent Application Publication
(10) Pub. No.: US 2010/0280328 A1
(43) Pub. Date: Nov. 4, 2010

(54) METHODS AND SYSTEMS FOR ILLUMINATION DURING PHLEBECTOMY PROCEDURES

(75) Inventors: Hoa D. Nguyen, San Jose, CA (US); Michael S. Mirizzi, San Jose, CA (US)

Correspondence Address: Tyco Healthcare Group L.P.;c/o I.P Legal Department Mark Kertz Esq., 15 Hampshire Street, Mansfield, MA, 02048, US

(73) Assignee: Tyco Healthcare Group, LP, Mansfield, MA (US)

(21) Appl. No.: 12/771,226

(22) Filed: Apr. 30, 2010

Related U.S. Application Data
(60) Provisional application No. 61/174,631, filed on May 1, 2009.

(51) Int. Cl. A61B 1/06

(52) U.S. Cl. 600/249

(57) ABSTRACT

A method comprising (a) illuminating a blood vessel in a relatively low-light surgical field by inserting a subcutaneous light source into tissue near the blood vessel and to a first location in the tissue underlying the blood vessel, and causing emission of light from the subcutaneous light source while the subcutaneous light source is at the first location, so that light from the subcutaneous light source passes through the tissue near the blood vessel, toward an observer; (b) drawing a portion of the tissue near the blood vessel through an incision in the skin; and (c) illuminating the drawn portion of tissue by activating an external light source in a hands-free manner, thereby facilitating determining whether the drawn tissue is the blood vessel.

### CROSS-REFERENCE TO RELATED APPLICATIONS

This application claims the benefit of priority under 35 U.S.C. §119(e) of U.S. Provisional Application No. 61/174,631 filed on May 1, 2009 and entitled “METHODS AND SYSTEMS FOR ILLUMINATION DURING PHLEBECTOMY PROCEDURES,” which is hereby incorporated herein by reference in its entirety and is to be considered a part of this specification.

### BACKGROUND

The disclosed embodiments relate generally to methods and systems for providing illumination during phlebectomy procedures. Phlebectomy is a standard treatment for varicosities arising from incompetent veins, particularly those below the saphenofemoral and saphenopopliteal junctions. This technique involves the avulsion and removal of the varicose veins through multiple stab incisions made directly over the veins, which are removed using surgical tools, such as hooks and mosquito forceps. Proper location of the veins to be removed is essential for proper situation of the incisions. In general, this is accomplished by preoperative vein location and marking, a procedure generally accomplished with the patient in a standing position so that the veins become engorged with blood due to the action of gravity and thus easily marked; however, the phlebectomy operation is generally conducted with the patient in a supine position. The position of the incompetent veins may shift during the transition from a standing to a supine position, and often further marking is conducted after the patient is placed in the surgical position in an attempt to track these shifts. Once the patient has been marked and incisions have been made along the length of the vein to be removed, the surgeon probes with the surgical implements, such as hooks, to locate the vein.

### SUMMARY

In one embodiment, a method comprises (a) illuminating a blood vessel in a relatively low-light surgical field by inserting a subcutaneous light source into tissue near the blood vessel and to a first location in the tissue underlying the blood vessel, and causing emission of light from the subcutaneous light source while the subcutaneous light source is at the first location, so that light from the subcutaneous light source passes through the tissue near the blood vessel, toward an observer; (b) drawing a portion of the tissue near the blood vessel through an incision in the skin; and (c) illuminating the drawn portion of tissue by activating an external light source in a hands-free manner, thereby facilitating determining whether the drawn tissue is the blood vessel.

In the method, the blood vessel can optionally be a vein, such as a varicose vein, or a superficial leg vein near the skin surface. The surgical field can optionally be an area at least about 20 cm wide and centered on the subcutaneous light source insertion site, extending at least about 20 cm from the skin surface at the subcutaneous light source insertion site toward an observer (e.g. a surgeon). The method can optionally further comprise, prior to drawing the portion of tissue near the vein, selecting the portion of the tissue based on observing the tissue illuminated by the subcutaneous light source. The external light source can optionally be worn on the surgeon's head and/or activated by a movement of the head (such as a nod of the head). The external light source can optionally be one which is activated without use of the hands or feet, and/or by the surgeon himself or herself without commanding another person to activate an external light source. The external light source can optionally provide broadband or white light in a floodlight fashion in the surgical field.

In another embodiment, a surgical illumination system comprises a subcutaneous illuminator having an elongate member configured for insertion into subcutaneous tissue. The elongate member has a light emitter near its distal end, and the light emitter is configured to emit light primarily in a generally radial direction, outward and away from a longitudinal axis of the elongate member. The system further comprises an external illuminator configured to illuminate a surgical field in a floodlight fashion. The external illuminator is configured for activation without use of the hands.

In the system, the external illuminator can optionally be configured to be worn on the surgeon's head and/or activated by a movement of the head (such as a nod of the head). The external illuminator can optionally include an inertial switch or accelerometer to facilitate such activation. The external illuminator can optionally be one which is activated without use of the hands or feet, and/or by the surgeon himself or herself without commanding another person to activate an external illuminator. The external illuminator can optionally provide broadband or white light in a floodlight fashion in the surgical field.

### BRIEF DESCRIPTION OF THE DRAWINGS

In the drawings:

FIG. 1 is a front view of an exemplary subcutaneous illumination apparatus of an illumination system according to one embodiment.

FIG. 2 is an enlarged view of the region labeled II in FIG. 1 showing an electroluminescent device of the subcutaneous illumination apparatus of FIG. 1.

FIG. 3 is a perspective view of an exemplary external illumination apparatus of the illumination system according to one embodiment.

FIG. 4 is a plan view of the external illumination apparatus of FIG. 3.

FIG. 5 is a perspective view of a switch according to one embodiment from the external illumination apparatus of FIG. 3.

FIG. 6 is a sectional view of the switch of FIG. 5 in an unactuated condition.

FIG. 7 is a sectional view similar to FIG. 6 with the switch in an actuated condition.

FIG. 8 is an exemplary electrical circuit for the external illumination apparatus of FIG. 3.

FIG. 9 illustrates the external illumination apparatus of FIG. 3 worn on the head of a user and actuation of the switch via movement of the head of the user.

### DETAILED DESCRIPTION

Methods and systems for providing illumination during phlebectomy procedures are disclosed. The methods and systems can provide subcutaneous illumination for visualization of veins and surrounding tissue and external illumination of material removed through the incisions for identification thereof. More particularly, after the patient has been marked and at least one incision has been made during the phlebectomy procedure, the surgeon attempts to locate the vein through the incisions, which can be difficult when the vein has shifted from the marked positions upon movement of the patient to the supine position. To facilitate vein location, the surgeon can employ an illumination device, such as a subcutaneous illumination device, that provides internal illumination for visually distinguishing the vein from the surrounding tissue. The subcutaneous illumination device can emit light at wavelengths selected to render the appearance of the vein dark, such as black, against a highlighted background of surrounding tissue. The illumination source can be relatively small and concentrated, and the region illuminated by the subcutaneous illumination device can often be viewed best when the room is relatively dark, which can be achieved by turning off or dimming the room lights. After location of the vein inside the body, the surgeon pulls a hook or other surgical implement from the incision and visually inspects material removed therewith for identification of the vein. However, this visual inspection can be difficult when the room is relatively dark for optimized efficacy of the subcutaneous illumination device, and the surgeon can employ an external illumination device that provides external illumination to facilitate the visual inspection.

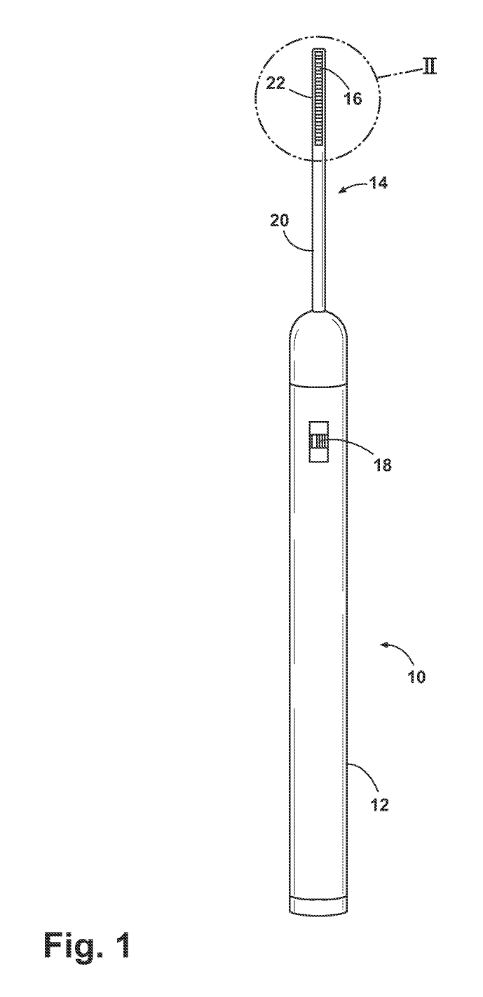

One embodiment of the system comprises a subcutaneous illumination apparatus 10, an example of which is illustrated in FIG. 1, and an external illumination apparatus 30, which will be described in detail below. Referring to FIG. 1, the exemplary subcutaneous illumination apparatus 10 comprises a handle 12 and an illuminator portion 14 housing an electroluminescent device 16. The handle 12 includes a switch 18 operatively coupled to the electroluminescent device 16 for selectively providing power to the electroluminescent device 16. The illuminator portion 14 comprises an elongated shaft 20 and a flattened blade or bladelike member 22 at the distal end of shaft 20. In certain embodiments, the shaft 20 can be of sufficient stiffness to permit the dissection of tissue or the avulsing of veins using the blade 22. The blade 22 can be sized for insertion into the subcutaneous surgical space through the small stab incisions typically employed in phlebectomy. The blade 22 can optionally be omitted and instead the shaft 22 can comprise a tube or cylinder of less than or equal to 7 French (2.33 mm) diameter along its length, with an insertable length of about 13-14 cm.

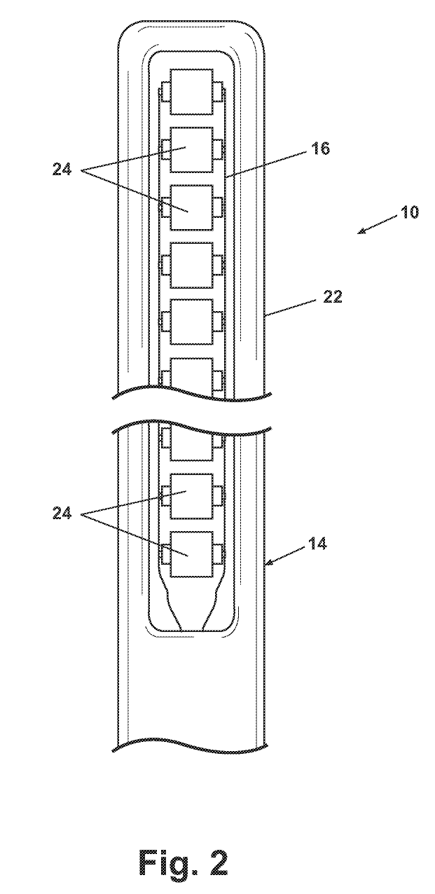

In the illustrated embodiment, the blade 22 or shaft 20 incorporates the electroluminescent device 16, which is configured to both generate and emit light in a generally radial direction, outward and away from a longitudinal axis of the illuminator portion 14. Any suitable type of electroluminescent lighting apparatus that can be accommodated within the blade 22 or shaft 20 can be employed as the electroluminescent device 16. Examples of electroluminescent devices include LED elements and other solid state light emitters. Individual light emitting elements 24 can be assembled into a linear array to form the device 16, as seen in the enlarged plan view of the electroluminescent device 16 in FIG. 2. In certain embodiments, the light emitting elements are configured to emit red or yellow light. In other embodiments, the light emitting elements are configured to emit light having a wavelength of less than 610 nm, or in the range of 630 to 670 nm. In other embodiments, the light emitting elements are configured to emit light substantially only in a wavelength or wavelength range that tends to highlight a blood vessel against surrounding tissue when the light is passed through the blood vessel and tissue. In other words, the incident light can cause the vein to appear darker than the surrounding tissue. The light emitting elements can be powered by batteries or other energy storage devices that are housed within the handle 12, or by an external power supply to which the apparatus 10 can be connected, such as a wall outlet.

The subcutaneous illumination apparatus 10 shown in FIGS. 1 and 2 and described above is one exemplary embodiment of the apparatus 10 that can be employed with the illumination system. Other types of subcutaneous illumination apparatuses can be employed with the illumination system and for the corresponding methods, including those described in U.S. Patent Application Publication No. 2007/0244371, published Oct. 19, 2007, and the corresponding U.S. patent application Ser. No. 11/732,771, filed Apr. 4, 2007, which are incorporated herein by reference in their entireties; the '371 publication is attached in the Appendix to the present application. Further, the system can employ other types of apparatuses for illuminating or otherwise identifying veins, such as a transdermal illumination apparatus (e.g. the VEINLITE™ available from TransLite LLC of Sugar Land, Tex.), in combination with or instead of the subcutaneous illumination apparatus.

As mentioned above, the illumination system comprises the external illumination apparatus 30 in addition to the subcutaneous illumination apparatus 10. While the subcutaneous illumination apparatus 10 provides internal illumination (i.e., illumination internal of the body of the patient) for identifying veins, the external illumination apparatus 30 provides external illumination (i.e., illumination external to the body of the patient) for inspection of the material removed from the patient's body. The external illumination apparatus 30 provides the external illumination without the use of the hands of the surgeon so that the hands remain free for manipulating the subcutaneous illumination apparatus 10, hooks, and/or other surgical instruments. The illustrated embodiment of the external illumination apparatus 30 achieves hands-free operation via mounting the apparatus 30 on the surgeon's head and actuation of the apparatus 30 in response to movement of the surgeon's head.

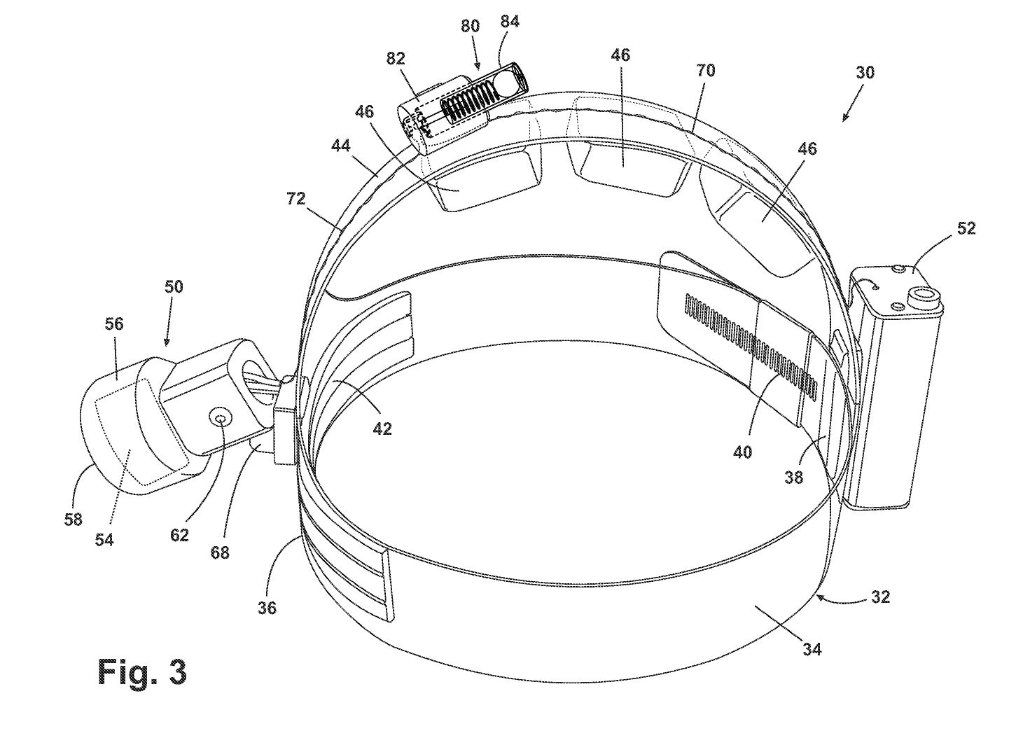

Referring now to FIG. 3, an exemplary embodiment of the external illumination apparatus 30 comprises a head mount 32 adapted to be worn on the head of the surgeon. The head mount 32 includes a generally circular headband 34 having a front portion 36 corresponding to the surgeon's forehead region, a rear portion 38 corresponding to the back of the surgeon's head, and an adjustable closure 40 for sizing the headband 34 according to the circumference of the head of the surgeon. The headband 34 can further incorporate one or more pads, such as a forehead pad 42 oriented for placement against the forehead that provide cushioning and facilitate a secure fit of the headband 34 around the head of the surgeon. The head mount 32 also comprises a harness 44 in the form of a band coupled to the front portion 36 and the rear portion 38 of the headband 34 and having an arcuate configuration so as to extend over the crown of the surgeon's head. Pads, such as crown pads 46, can be positioned on the underside of the harness 50 to facilitate a comfortable fit of the harness 44 against the crown of the surgeon's head.

The headband 34 supports an illumination assembly 50 at the front portion 36 and a power source 52 for the illumination assembly 50 at the rear portion 38. The illumination assembly 50 comprises an illumination source 54 provided within an illumination source housing 56 and a lens 58 positioned at the front end of the housing 56. The lens 58 protects the illumination source 54 within the housing 56 and can optionally function as a filter for light emitted by the illumination source 54. An adjustable mount 60 couples the housing 56 to the headband 34 and includes a pivotable joint 62 at which the housing 56 connects to the mount 60 and about which the housing 56 can pivot for moving the housing 56 and, thereby, the illumination source 54, in up and down directions. In some embodiments, the mount 60 can be configured for additional adjustment of the housing 56 and/or the illumination source 54 in other directions, such as lateral or side-to-side movement. In certain embodiments, the mount 60 can be configured for adjustment of the housing 56 and/or the illumination source 54 in any direction, such as via a universal or U-joint. The adjustment of the housing 54 and/or the illumination source 54 in the illustrated embodiment is accomplished manually, and other embodiments employ other adjustment methods, such as verbal/speech recognition adjustment to further render the external illumination apparatus 30 hands-free.

The illumination source 54 can be any suitable device that provides desired external illumination during a phlebectomy procedure. In one embodiment, the desired external illumination is a broad field of white light that sufficiently illuminates a relatively dark room to allow the surgeon to perform the visual inspection of the material removed from the patient. Such light can be provided by any suitable illumination source, including, but not limited to, electroluminescent illumination sources, such as LEDs, and conventional light bulbs, such as fluorescent and incandescent light bulbs. In other embodiments, the desired external illumination can be a broad field of colored (i.e., not white) light that facilitates the visual inspection of the material or a narrow, focused field of white or colored light. In one embodiment, the wavelength(s) of light provided by the illumination source 54 can be selected to aid the surgeon in distinguishing the vein from surrounding tissues, such as fat tissue.

The power source 52 that provides power to the illumination source 54 in the illustrated embodiment comprises a portable power source in the form of a battery. The battery can be any suitable battery, including replaceable and/or rechargeable batteries. In one embodiment, the battery can be rechargeable and removed from the head mount 32 for coupling to a charger. In another embodiment, the entire external illumination apparatus 30 can be coupled to a charger, such as via a docking station or a cord that connects the battery to a power outlet. The power source 52 can also include a cord or other physical connection that couples the illumination source 54 to an external source of power, either to replace the portable power source or to be used as a back-up in the event that the portable power source becomes depleted during use of the external illumination apparatus 30.

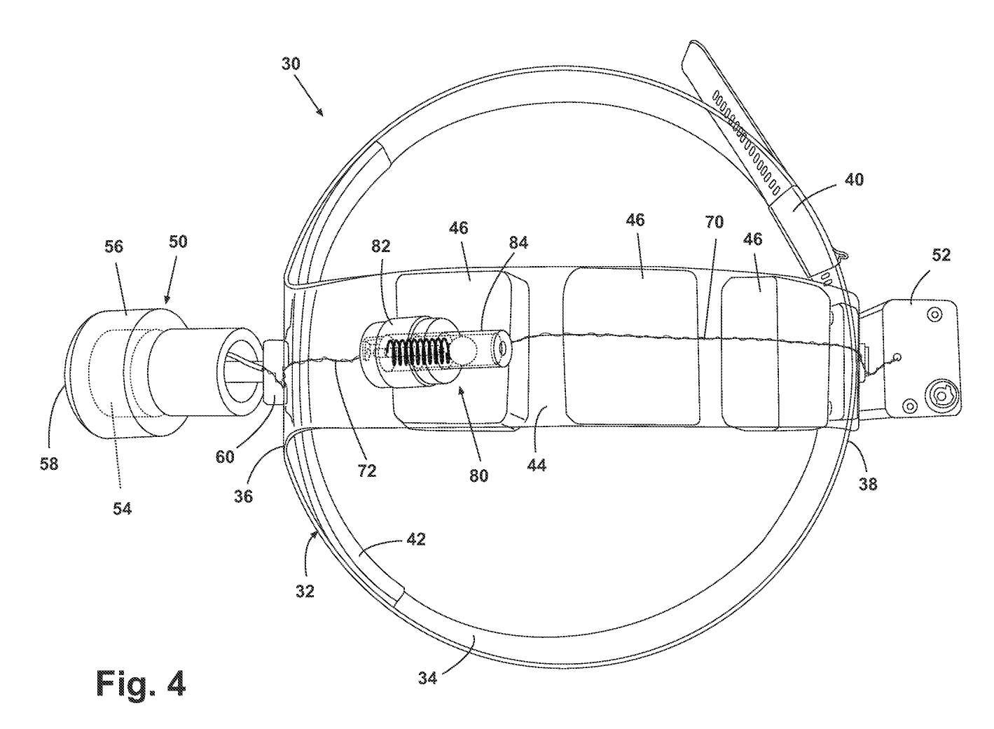

A set of wires 70, 72 electrically connects the power source 52 to the illumination source 54, and a switch 80 controls the supply of electricity from the power source 52 to the illumination source 54. As best viewed in FIG. 4, which is a top view of the external illumination apparatus 30, the harness 44 of the head mount 32 supports the wires 70, 72 and the switch 80. The wire 70 electrically couples the power source 52 to the switch 80, and the wire 72 electrically couples the switch 80 to the illumination source 54.

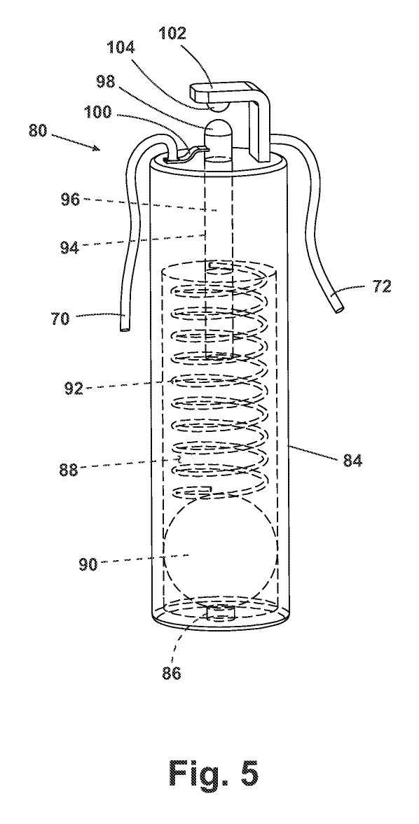

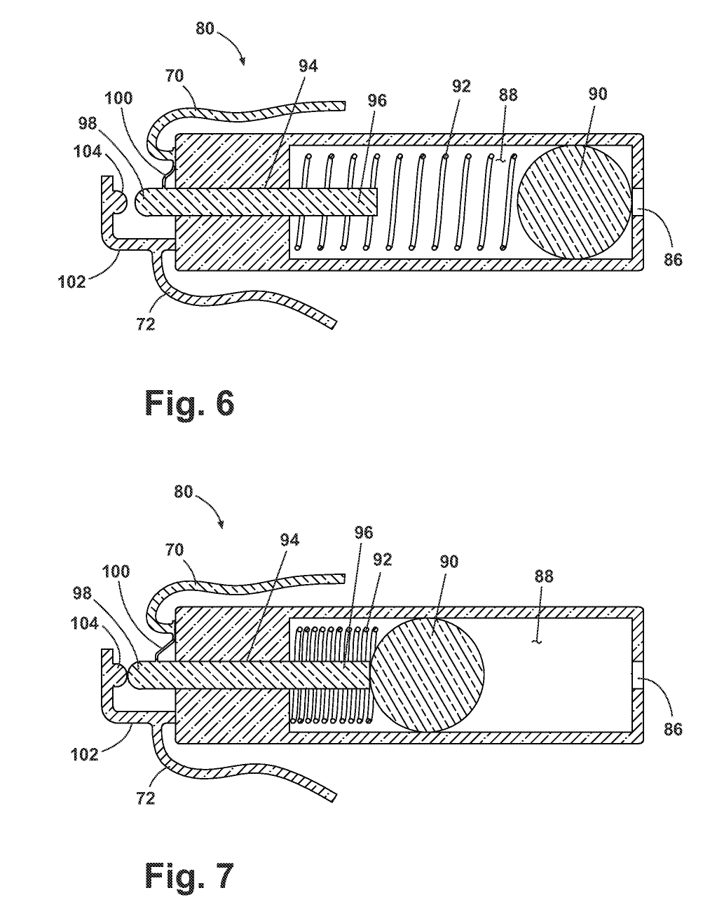

In the illustrated embodiment, the switch 80 is an inertial switch in the form of an acceleration switch responsive to the movement of the surgeon's (or other wearer's) head. The switch 80 is partially located within a switch housing 82 mounted to the harness 44. Referring now to FIG. 5, which is a perspective view of the switch 80, and FIG. 6, which is a sectional view bisecting the switch 80 along its longitudinal axis, the switch 80 comprises a casing 84 having, at its rear end, a vent 86 for an interior chamber 88 that receives a ball 90 and a compression spring 92 and, at its front end, a bore 94 that slidably receives a plunger 96. The plunger 96 extends from within the chamber 88 and through the bore 94 to project beyond the front end of the casing 84. The compression spring 92 surrounds the portion of the plunger 96 within the chamber 88 and spaces the ball 90 from the plunger 96 when the compression spring 92 is in its natural extended condition shown in FIGS. 5 and 6. The front end of the plunger 96 functions as a first electrical contact 98 and is electrically coupled to the wire 70 by a conductive leaf spring 100. The switch 80 further includes an L-shaped arm 102 extending from the front end of the casing 84 and terminating at a second electrical contact 104 linearly aligned with and facing the first electrical contact 98. The conductive arm 102 couples the wire 72 with the second electrical contact 104. The switch 80 is normally in an unactuated condition where the compression spring 92 is in its extended condition, and the plunger 96 is in a retracted position such that the first and second electrical contacts 98, 104 are spaced from each other with no electrical communication therebetween, as shown in FIGS. 5 and 6.

Actuation of the switch 80 results from movement of the ball 90 within the chamber 88. When the switch 80 undergoes sufficient movement for the ball 90 to accelerate forward and overcome the bias of the compression spring 92, the moving ball 90 compresses the compression spring 92 and eventually contacts the plunger 96. The moving ball 90 applies a linear force to the plunger 96, which responds by moving linearly through the bore 94 to an extended position whereby the first electrical contact 98 on the plunger 96 contacts the second electrical contact 104 on the arm 102, as illustrated in FIG. 7, for momentarily closing an electrical circuit, which is described in detail below. The compression spring 92 at this point is completely or near completely compressed, and the forward movement of the ball 90 ceases. Because the forward acceleration of the ball 90 terminates, the compression spring 92 expands and returns to its extended condition, thereby pushing the ball 90 to the rear end of the chamber 88 and spacing the ball 90 from the plunger 96. Upon removal of the linear force applied by the ball 90, the plunger 96 slides rearward within the bore 94 due to the bias of the leaf spring 100 and returns to its retracted position. The retraction of the plunger 96 spaces the first electrical contact 98 from the second electrical contact 104, thereby opening the electrical circuit.

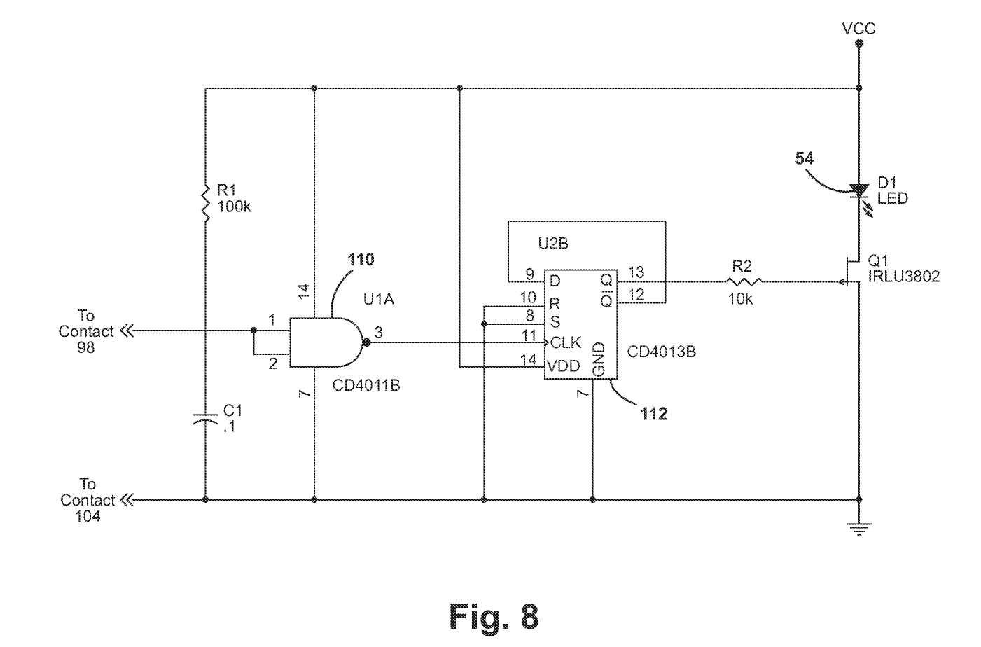

The electrical circuit can be any suitable electrical circuit that functions to turn the illumination source 54 on and off upon actuation of the switch 80, and FIG. 8 illustrates a diagram of an exemplary embodiment of the electrical circuit. The switch 80 is a momentary switch implemented, by example, through a NAND gate 110, such as a CD4011B gate, and a D-type flip-flop 112, such as a CD4013B flip-flop. When the contacts 98, 104 are closed upon actuation of the switch 80, the output at pin 3 of the gate 110 is sent to the input CLK at pin 11 of the flip-flop 112. At an initial state with the illumination source 54 off, the input D at pin 9 of the flip-flop 112 and the output Q-bar at pin 12 are HIGH. When the contacts are closed, the input CLK at pin 11 goes from HIGH to LOW. Data from the input D at pin 9 is shifted to output Q at pin 13, which makes the output Q at pin 13 HIGH and turns the illumination source 54 on and makes Q-bar at pin 12 of the flip-flop 112 LOW. The next actuation of the switch 80 shifts the data from the input D at pin 9, which is now LOW, to the output Q at the pin 13 to turn the illumination source 54 off. This cycle repeats with subsequent actuations of the switch 80.

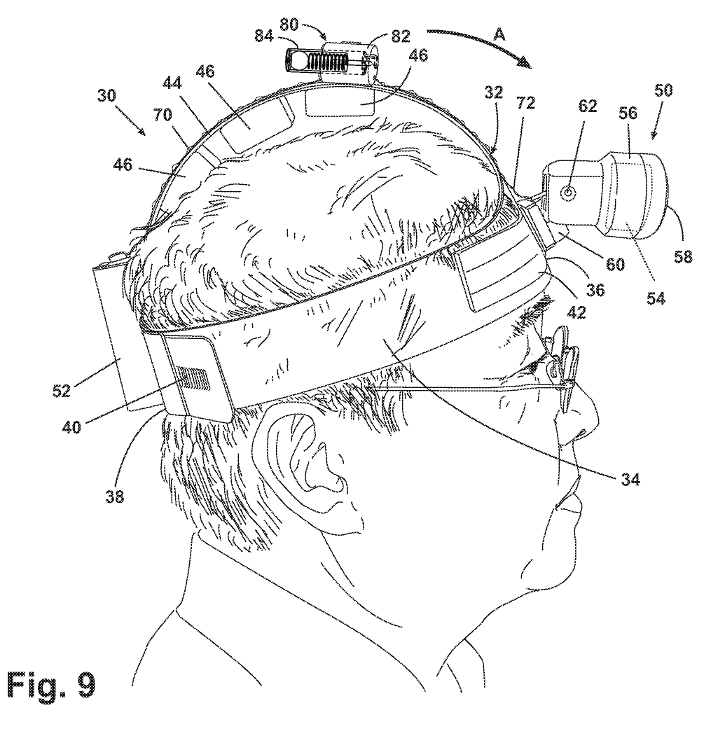

Referring now to FIG. 9, which shows the external illumination apparatus 30 worn on the head of a surgeon, the switch 80 in the illustrated embodiment is positioned forward of the apex of the harness 44 to facilitate actuation of the switch 80. The switch position preferably corresponds to forward of the head apex when the external illumination apparatus 30 is worn on the surgeon's head. In this position, the switch 80 is slightly angled with the ball 90 behind the compression spring 92. Actuation of the switch 80 occurs when the surgeon quickly nods the head, as indicated by the arrow “A” in FIG. 9. Forward and downward movement of the head during the nod (together with a relatively sudden stop and/or momentary reversal of the direction of movement of the head) induces forward movement of the ball 90 against the compression spring 92. Because the ball 90 is angled, gravity aids forward movement of the ball 90 within the chamber 88 against the compression spring 92. While the switch 80 has been described and illustrated as being located forward of the harness apex, the switch 80 can be positioned at any desired location on the head mount 32.

As just described, movement of the head of the surgeon wearing the external illumination apparatus 30 actuates the switch 80 to turn the illumination source 54 on and off. The switch 80 defaults to the unactuated condition shown in FIGS. 5 and 6 and must be converted to the actuated condition shown in FIG. 7 via a quick head nod or other suitable motion to turn the illumination source 54 on or off In particular, to actuate the switch 80, the surgeon performs a head nod, whereby the ball 90 moves forward to compress the compression spring 92 and push the plunger 96 through the bore 94 and against the bias of the leaf spring 100 to establish momentary contact between the first and second electrical contacts 98, 104. If the illumination source 54 is off prior to the actuation of the switch 80, then the output Q at pin 13 of the flip-flop 112 in the electrical circuit becomes HIGH upon contact between the first and second electrical contacts 98, 104, as described above, to turn the light on. On the other hand, if the illumination source 54 is on prior to the actuation of the switch 80, then the output Q at pin 13 of the flip-flop 112 in the electrical circuit becomes LOW upon contact between the first and second electrical contacts 98, 104, as described above, to turn the light off After the momentary contact between the first and second electrical contacts, 98, 104, the compression spring 92 pushes the ball 90 rearward, and the leaf spring 100 retracts the plunger 96, as described above, to return the switch 80 to the unactuated condition. In short, the surgeon quickly nods to turn the illumination source 54 on and quickly nods again to turn the illumination source 54 off.

An exemplary method of operation of the above described embodiment of the system for providing illumination during phlebectomy procedures follows. The method comprises providing internal illumination with the subcutaneous illumination apparatus 10 and providing external illumination with the external illumination apparatus 30.

Commonly, phlebectomy procedures involve the use of tumescent anesthesia, using, for example, large-volume, low-concentration lidocaine. Subcutaneous application of the tumescing solution elevates the veins closer to the skin surface and increases the field of illumination. Where the standard anesthesia protocol is inadequate to provide the desired conditions for illumination, additional saline solution can be injected.

The surgeon's preparation for the phlebectomy procedure includes placing the external illumination apparatus 30 upon the head by positioning the headband 34 around the head with the front portion 36 on the forehead and the rear portion 38 on the rear of the head such that the illumination assembly 50 and power source 52 are located on the forehead and the rear of the head, respectively, as shown in FIG. 9. Adjustment of the headband 34 can be accomplished by adjusting the closure 40. If needed, the surgeon can adjust the position of the illumination assembly 50 so that the light from the illumination source 54 projects in a desired direction.

During the following portion of the method involving the use of the subcutaneous illumination apparatus 10, the illumination source 54 of the external illumination apparatus 30 is in the off condition. At the least, the illumination source 54 is in the off condition when the subcutaneous illumination apparatus 10 illuminates the interior of the body for vein identification. Further, lights in the procedure room, such as an operating room, are preferably off or dimmed for optimized performance of the subcutaneous illumination apparatus 10.

Holding the handle 12 of the apparatus 10, the surgeon inserts the illuminator portion 14 through a skin incision made in the vicinity of a vein to be avulsed. The surgeon can engage the electroluminescent device 16 using the switch 18 either before inserting the illuminator portion 14 or after insertion. The handle 12 is manipulated to place the apparatus 10 beneath a vein to be avulsed, with the electroluminescent device 16 facing upward (or radially outward, or otherwise toward the surgeon or observer). The light emitted by the electroluminescent device 16 passes upward through the vein (including the deoxygenated blood in the vein) and the surrounding tissue, thereby enabling the surgeon to better visualize the vein. For example, where the electroluminescent device 16 emits red, yellow or amber light, or light having a wavelength of 630-670 nm, or approximately 610 nm or less, the vein appears black in contrast to the surrounding tissue, which appears red or yellow. When viewed through the skin, the vein appears as a dark shadow, thereby facilitating location and the placement of further incisions, if required.

For the initial location of the vein, in one embodiment, the surgeon orients the longitudinal axis of the electroluminescent device 16 transverse to the presumed longitudinal axis of the vein. This configuration offers an improved chance that the vein falls within the field of illumination of the electroluminescent device 14. Once the vein has been located, the surgeon can employ the blade 22 to locally avulse the vein prior to its removal through the skin incisions. Alternatively, a separate surgical implement such as a phlebectomy hook or forceps can be inserted into the incision(s) and employed to hook or grip, and avulse the vein along its length while it is being visualized through the skin.

When the surgeon removes the vein through the incision, some other material, such as fat and other surrounding tissues, is frequently removed along with or instead of the vein, and the surgeon performs a visual inspection of the removed material for identification of the vein and separation of the vein from the surrounding tissue. Because the procedure room is relatively dark, the surgeon can employ the external illumination apparatus 30 to provide illumination for the visual inspection. In particular, the surgeon, wearing the external illumination apparatus 30 on the head, quickly nods the head to actuate the switch 80, as described above in detail, to convert the illumination source 54 from the off condition to the on condition. The light provided from the illumination source 54 enables the surgeon to better visualize the removed material for more accurate identification of the removed vein. If surrounding tissue has indeed been removed with the vein, the surgeon can separate the vein from the surrounding tissue. Upon completion of the visual inspection or whenever desired, the surgeon performs another quick head nod to actuate the switch 80 and, thereby, convert the illumination source 54 from the on condition to the off condition. The surgeon can turn the illumination source 54 on and off as needed throughout the phlebectomy procedure.

The above method can be adapted for use with other types of apparatuses that provide internal illumination. Examples of other operation methods for other types of internal illumination apparatuses are given in the aforementioned and incorporated '371 publication and corresponding application. Further, the above method can be adapted for use with other types of switches that are activated in hands-free (or hands-free and feet-free) manners other than nodding the head.

While the external illumination apparatus 30 has been described as part of the illumination system for phlebectomy procedures, the apparatus 30 can be employed alone or in other systems for other types of procedures. Further, the external illumination apparatus 30 can be employed with other types of internal illumination devices for phlebectomy or other types of procedures.

While certain invention(s) have been specifically described herein in connection with certain specific embodiments thereof, it is to be understood that this description is an illustration of useful embodiments of the invention(s) and not a limitation of the scope of the invention(s).

What is claimed is:

1. A method, comprising:
illuminating a blood vessel in a relatively low-light surgical field by inserting a subcutaneous light source into tissue near the blood vessel and to a first location in the tissue underlying the blood vessel, and causing emission of light from the subcutaneous light source while the subcutaneous light source is at the first location, so that light from the subcutaneous light source passes through the tissue near the blood vessel, toward an observer;
drawing a portion of the tissue near the blood vessel through an incision in the skin; and
illuminating the drawn portion of tissue by activating an external light source in a hands-free manner, thereby facilitating determining whether the drawn tissue is the blood vessel.

2. The method of claim 1, wherein the blood vessel is a vein.

3. The method of claim 1, wherein the blood vessel is a varicose vein.

4. The method of claim 1, wherein the blood vessel is a superficial leg vein.

5. The method of claim 1, wherein the surgical field is an area at least about 20 cm wide and centered on the subcutaneous light source insertion site, extending at least about 20 cm from the skin surface at the subcutaneous light source insertion site toward the surgeon.

6. The method of claim 1, further comprising:
prior to drawing the portion of tissue near the blood vessel, selecting the portion of the tissue based on observing the tissue illuminated by the subcutaneous light source.

7. The method of claim 1, wherein the external light source is worn on the surgeon's head.

8. The method of claim 1, wherein the external light source is activated by a movement of the head.

9. The method of claim 8, wherein the external light source is activated by a nod of the head.

10. The method of claim 1, wherein the external light source is activated without use of the hands or feet.

11. The method of claim 10, wherein the external light source is activated by the surgeon himself or herself without commanding another person to activate an external light source.

12. The method of claim 1, wherein the external light source provides broadband or white light in a floodlight fashion in the surgical field.

13. A surgical illumination system, comprising:
a subcutaneous illuminator having an elongate member configured for insertion into subcutaneous tissue, the elongate member having a light emitter near its distal end, the light emitter being configured to emit light primarily in a generally radial direction, outward and away from a longitudinal axis of the elongate member; and
an external illuminator configured to illuminate a surgical field in a floodlight fashion, the external illuminator being configured for activation without use of the hands.

14. The system of claim 13, wherein the external illuminator is configured to be worn on the surgeon's head.

15. The system of claim 14, wherein the external illuminator is configured to be activated by a movement of the head.

16. The system of claim 15, wherein the external illuminator is configured to be activated by a nod of the head.

17. The system of claim 15, wherein the external illuminator includes an inertial switch or accelerometer to facilitate activation by a movement of the head.

18. The system of claim 13, wherein the external illuminator is configured to be activated without use of the hands or feet.

19. The system of claim 13, wherein the external illuminator is configured to be activated by the surgeon himself or herself without commanding another person to activate an external illuminator.

20. The system of claim 13, wherein the external illuminator provides broadband or white light in a floodlight fashion in the surgical field.
